SEQUENCE LISTING (66 sequences)

SEQ ID NO: 1 - RNA, 22 nt, Homo Sapiens
accacugaccguugacuguacc

SEQ ID NO: 2 - RNA, 21 nt, Homo Sapiens
aggcccuguccucugccccag

SEQ ID NO: 3 - RNA, 22 nt, Homo Sapiens
acccuaucaauauugucucugc

SEQ ID NO: 4 - RNA, 22 nt, Homo Sapiens
aggccuguggcuccucccucag

SEQ ID NO: 5 - RNA, 21 nt, Homo Sapiens
aucacauugccagggauuacc

SEQ ID NO: 6 - RNA, 19 nt, Homo Sapiens
ggcuggucagaugggagug

SEQ ID NO: 7 - RNA, 24 nt, Homo Sapiens
acccccgggcaaagaccugcagau

SEQ ID NO: 8 - RNA, 20 nt, Homo Sapiens
ucccacuacuucacuuguga

SEQ ID NO: 9 - RNA, 21 nt, Homo Sapiens
cuaccccccaucccccuguag

SEQ ID NO: 10 - RNA, 21 nt, Homo Sapiens
ucaagugucaucugucccuag

SEQ ID NO: 11 - RNA, 22 nt, Homo Sapiens
aaggagcucacagucuauugag

SEQ ID NO: 12 - RNA, 23 nt, Homo Sapiens
aacauucauugcugucggugggu

SEQ ID NO: 13 - RNA, 21 nt, Homo Sapiens
cugacuguugccguccuccag

SEQ ID NO: 14 - RNA, 22 nt, Homo Sapiens
aguugccagggcugccuuuggu

SEQ ID NO: 15 - RNA, 22 nt, Homo Sapiens
ugcuggaucagugguucgaguc

SEQ ID NO: 16 - RNA, 23 nt, Homo Sapiens
ccuccguguuaccuguccucuag

SEQ ID NO: 17 - RNA, 20 nt, Homo Sapiens
ggcuccuuggucuaggggua

SEQ ID NO: 18 - RNA, 22 nt, Homo Sapiens
uccccuucugcaggccugcugg

SEQ ID NO: 19 - RNA, 22 nt, Homo Sapiens
ucuucucuguuuuggccaugug

SEQ ID NO: 20 - RNA, 22 nt, Homo Sapiens
ucugugcuucaccccuacccag

SEQ ID NO: 21 - RNA, 22 nt, Homo Sapiens
agccaggcucugaagggaaagu

SEQ ID NO: 22 - RNA, 22 nt, Homo Sapiens
caaccucgacgaucuccucagc

SEQ ID NO: 23 - RNA, 23 nt, Homo Sapiens
uggcugcuucccuuggucuccag

SEQ ID NO: 24 - RNA, 22 nt, Homo Sapiens
gggagccaggaaguauugaugu

SEQ ID NO: 25 - RNA, 22 nt, Homo Sapiens
ugagguaguagauuguauaguu

SEQ ID NO: 26 - RNA, 21 nt, Homo Sapiens
aagccucuguccccaccccag

SEQ ID NO: 27 - RNA, 22 nt, Homo Sapiens
ucggauccgucugagcuuggcu

SEQ ID NO: 28 - RNA, 22 nt, Homo Sapiens
cguguauuugacaagcugaguu

SEQ ID NO: 29 - RNA, 21 nt, Homo Sapiens
uaacgcauaauauggacaugu

SEQ ID NO: 30 - RNA, 22 nt, Homo Sapiens
aaucagugaaugccuugaaccu

SEQ ID NO: 31 - RNA, 22 nt, Homo Sapiens
uugucucuuguuccucacacag

SEQ ID NO: 32 - RNA, 21 nt, Homo Sapiens
cuccguuugccuguuucgcug

SEQ ID NO: 33 - RNA, 22 nt, Homo Sapiens
uauguaacaugguccacuaacu

SEQ ID NO: 34 - RNA, 22 nt, Homo Sapiens
uauguaacacgguccacuaacc

SEQ ID NO: 35 - RNA, 23 nt, Homo Sapiens
cagugcaaugauauugucaaagc

SEQ ID NO: 36 - RNA, 21 nt, Homo Sapiens
ugacuucuaccucuuccaaag

SEQ ID NO: 37 - RNA, 21 nt, Homo Sapiens
uccgguucucagggcuccacc

SEQ ID NO: 38 - RNA, 22 nt, Homo Sapiens
ucagguguggaaacugaggcag

SEQ ID NO: 39 - RNA, 22 nt, Homo Sapiens
uuugaggcuacagugagaugug

SEQ ID NO: 40 - RNA, 22 nt, Homo Sapiens
uucucuuucuuuagccuugugu

SEQ ID NO: 41 - RNA, 22 nt, Homo Sapiens
uuaauuuuuuguuucggucacu

SEQ ID NO: 42 - RNA, 22 nt, Homo Sapiens
ugaaggucuacugugugccagg

SEQ ID NO: 43 - RNA, 22 nt, Homo Sapiens
uagcaagagaaccauuaccauu

SEQ ID NO: 44 - RNA, 22 nt, Homo Sapiens
caaaaaccggcaauuacuuuug

SEQ ID NO: 45 - RNA, 22 nt, Homo Sapiens
uuagccaauuguccaucuuuag

SEQ ID NO: 46 - RNA, 22 nt, Homo Sapiens
gcuggugcaaaaguaauggcgg

SEQ ID NO: 47 - RNA, 23 nt, Homo Sapiens
agguuacccgagcaacuuugcau

SEQ ID NO: 48 - RNA, 21 nt, Homo Sapiens
ccggccgccggcuccgccccg

SEQ ID NO: 49 - RNA, 22 nt, Homo Sapiens
auccgcgcucugacucucugcc

SEQ ID NO: 50 - RNA, 22 nt, Homo Sapiens
uuuaggauaagcuugacuuuug

SEQ ID NO: 51 - RNA, 21 nt, Homo Sapiens
caucccuugcaugguggaggg

SEQ ID NO: 52 - RNA, 22 nt, Homo Sapiens
aaaaguaauugugguuuuggcc

SEQ ID NO: 53 - RNA, 22 nt, Homo Sapiens
aacucugaccccuuagguugau

SEQ ID NO: 54 - RNA, 22 nt, Homo Sapiens
aggccuguggcuccucccucag

SEQ ID NO: 55 - RNA, 22 nt, Homo Sapiens
ugugacaauagagaugaacaug

SEQ ID NO: 56 - RNA, 21 nt, Homo Sapiens
ucuugaagucagaacccgcaa

SEQ ID NO: 57 - RNA, 22 nt, Homo Sapiens
caaaaguaauuguggauuuugu

SEQ ID NO: 58 - RNA, 23 nt, Homo Sapiens
ucuggcaaguaaaaaacucucau

SEQ ID NO: 59 - RNA, 23 nt, Homo Sapiens
aucaacagacauuaauugggcgc

SEQ ID NO: 60 - RNA, 22 nt, Homo Sapiens
uaggggaaaaguccugauccgg

SEQ ID NO: 61 - RNA, 22 nt, Homo Sapiens
cucgugggcucuggccacggcc

SEQ ID NO: 62 - RNA, 22 nt, Homo Sapiens
ucugugcuucaccccuacccag

SEQ ID NO: 63 - RNA, 21 nt, Homo Sapiens
cggggcagcucaguacaggau

SEQ ID NO: 64 - RNA, 23 nt, Homo Sapiens
uggaagacuagugauuuuguugu

SEQ ID NO: 65 - RNA, 25 nt, Homo Sapiens
aaaaaccacaauuacuuuugcacca

SEQ ID NO: 66 - RNA, 22 nt, Homo Sapiens
uuuugcaauauguuccugaaua

(19) United States
(12) Patent Application Publication
(10) Pub. No.: US 2017/0130269 A1
(43) Pub. Date: May 11, 2017

(54) DIAGNOSIS OF NEUROMYELITIS OPTICA VS. MULTIPLE SCLEROSIS USING MIRNA BIOMARKERS

(71) Applicant: SIEMENS HEALTHCARE GMBH, Erlangen (DE)

(72) Inventors: Andreas KELLER, Puettlingen (DE); Jan KIRSTEN, Bamberg (DE); Cord Friedrich STAEHLER, Hirschberg an der Bergstrasse (DE); Christina BACKES, Saarbruecken (DE); Petra LEIDINGER, Wadern (DE); Eckart MEESE, Huetschenhausen (DE)

(21) Appl. No.: 15/322,531

(22) PCT Filed: Jun. 24, 2015
(86) PCT No.: PCT/EP2015/064213

§ 371 Date: Dec. 28, 2016

(30) Foreign Application Priority Data
Jun. 30, 2014 (EP) 14175006.7

(51) Int. Cl. C12Q 1/68

(52) U.S. Cl. CPC C12Q 1/6883; C12Q 2600/158; C12Q 2600/178; C12Q 2600/118; C12Q 2600/16

(57) ABSTRACT

The invention relates to a method of diagnosis of neuromyelitis optica, in particular of differential diagnosis of neuromyelitis optica (NMO) vs. multiple sclerosis (MS) using miRNA biomarkers. These miRNAs could potentially serve as future diagnostic biomarkers for NMO and help in diagnosis, monitoring disease activity, and evaluation of treatment responses in patients with MS.

### PRIORITY STATEMENT

This application is the national phase under 35 U.S.C. §371 of PCT International Application No. PCT/EP2015/064213 which has an International filing date of 24 Jun. 2015, which designated the United States of America and which claims priority to European patent application number 14175006.7 filed 30 Jun. 2014, the entire contents of which are hereby incorporated herein by reference.

### REFERENCE TO A SEQUENCE LISTING

The material in the ASCII text file entitled “3081193_1.txt” is hereby incorporated by reference in its entirety pursuant to 37 C.F.R. §1.52(e)(5). The ASCII text file entitled “3081193_1.txt” was created on 30 Jun. 2014 and the size is 9 KB.

### FIELD

The invention relates to a method of diagnosis of neuromyelitis optica, in particular of differential diagnosis of neuromyelitis optica (NMO) vs. multiple sclerosis (MS) using miRNA biomarkers.

### BACKGROUND

Very recently, molecular diagnostics has increasingly gained in importance. It has found an entry into the clinical diagnosis of diseases (inter alia detection of infectious pathogens, detection of mutations of the genome, detection of diseased cells and identification of risk factors for predisposition to a disease).

In particular, through the determination of gene expression in tissues, nucleic acid analysis opens up very promising new possibilities in the study and diagnosis of disease.

Nucleic acids of interest to be detected include genomic DNA, expressed mRNA and other RNAs such as MicroRNAs (abbreviated miRNAs). MiRNAs are a new class of small RNAs with various biological functions (A. Keller et al., Nat Methods. 2011 8(10):841-3). They are short (average of 20-24 nucleotide) ribonucleic acid (RNA) molecules found in eukaryotic cells. Several hundred different species of microRNAs (i.e. several hundred different sequences) have been identified in mammals. They are important for post-transcriptional gene regulation and bind to complementary sequences on target messenger RNA transcripts (mRNAs), which can lead to translational repression or target degradation and gene silencing. As such they can also be used as biologic markers for research, diagnosis and therapy purposes.

Neuromyelitis optica (NMO) is a rare disorder, which resembles multiple sclerosis (MS) in several ways, but requires a different course of treatment. Both diseases have similar symptoms, but NMO patients cannot be diagnosed using McDonald criteria and magnetic resonance tomography. Further there are no validated biomarkers allowing a differentiation between NMO and MS

Diagnosis is initially a clinical diagnosis, i.e. based on health survey (anamnesis) and neurological examinations, by looking for symptoms of optic neuritis and spinal cord involvement while symptoms that are due to lesions in the brain are excluded. To confirm the diagnosis, the determination of aquaporin—4 antibodies and magnetic resonance imaging of the skull and spine are necessary and for the differential diagnosis, a lumbar puncture, evoked potentials and possibly electromyography/neurography.

A safe differentiation of NMO and MS is not always possible at the beginning of the disease. If a patient develops additionally symptoms that point to an involvement of the brain outside of the optic nerves, this would call the diagnosis in question. Another syndrome, retrobulbar neuritis, can affect the vision as well, but by definition remains without spinal cord involvement.

Multiple sclerosis (MS) is an inflammatory autoimmune disease of the central nervous system, in which the myelin sheaths around the axons of the brain and spinal cord are damaged, leading to demyelination and scarring as well as a broad spectrum of clinical signs and symptoms. MS can be classified into different disease subtypes, including relapsing/remitting MS (RRMS), secondary progressive MS, primary progressive MS, progressive relapsing MS. The relapsing-remitting subtype is characterized by unpredictable relapses followed by periods of months to years of relative quiet (remission) with no new signs of disease activity. The relapsing-remitting subtype usually begins with a clinically isolated syndrome (CIS). In CIS, a patient has an attack suggestive of demyelination. Often CIS marks the onset of MS.

The diagnosis of multiple sclerosis usually involves analysis of different clinical data, imaging data, and laboratory data. Some patients live years with MS before receiving a diagnosis of disease.

Therefore, there exists an unmet need for an efficient, simple, reliable diagnostic test for NMO, in particular for a diagnostic test which can differentiate between NMO and MS.

### SUMMARY

The technical problem underlying the present invention is to provide biological markers allowing for diagnosis of multiple sclerosis, predict the risk of developing multiple sclerosis, or predict an outcome of multiple sclerosis.

### DETAILED DESCRIPTION

Before the invention is described in detail, it is to be understood that this invention is not limited to the particular component parts of the process steps of the methods described as such methods may vary. It is also to be understood that the terminology used herein is for purposes of describing particular embodiments only, and is not intended to be limiting. It must be noted that, as used in the specification and the appended claims, the singular forms “a,” “an” and “the” include singular and/or plural referents unless the context clearly dictates otherwise. It is also to be understood that plural forms include singular and/or plural referents unless the context clearly dictates otherwise. It is moreover to be understood that, in case parameter ranges are given which are delimited by numeric values, the ranges are deemed to include these limitation values.

### DEFINITIONS

Unless defined otherwise, technical and scientific terms used herein have the same meaning as commonly understood by one of ordinary skill in the art to which this invention belongs.

The term “neuromyelitis optica” or “NMO” as used herein relates to a recurrent inflammatory and demyelinating disease of the optical nerve and spinal cord involving and is meant to include all clinical stages and subtypes of disease. It is also commonly referred to as Devic's disease.

The term “multiple sclerosis” or “MS” as used herein relates to an inflammatory disease of the nervous system and is meant to include all clinical stages and subtypes of disease, including clinically isolated symptoms of MS (CIS), relapsing/remitting MS (RRMS), secondary progressive MS, primary progressive MS, and progressive relapsing MS.

The term “predicting an outcome” of a disease, as used herein, is meant to include both a prediction of an outcome of a patient undergoing a given therapy and a prognosis of a patient who is not treated.

An “outcome” within the meaning of the present invention is a defined condition attained in the course of the disease. This disease outcome may e.g. be a clinical condition such as “relapse of disease”, “remission of disease”, “response to therapy”, a disease stage or grade or the like.

A “risk” is understood to be a probability of a subject or a patient to develop or arrive at a certain disease outcome. The term “risk” in the context of the present invention is not meant to carry any positive or negative connotation with regard to a patient's wellbeing but merely refers to a probability or likelihood of an occurrence or development of a given event or condition.

The term “clinical data” relates to the entirety of available data and information concerning the health status of a patient including, but not limited to, age, sex, weight, menopausal/hormonal status, etiopathology data, anamnesis data, data obtained by in vitro diagnostic methods such as blood or urine tests, data obtained by imaging methods, such as x-ray, computed tomography, MRI, PET, spect, ultrasound, electrophysiological data, genetic analysis, gene expression analysis, biopsy evaluation, intraoperative findings.

The term “classification of a sample” of a patient, as used herein, relates to the association of said sample with at least one of at least two categories. These categories may be for example “high risk” and “low risk”, high, intermediate and low risk, wherein risk is the probability of a certain event occurring in a certain time period, e.g. occurrence of disease, progression of disease, etc. It can further mean a category of favorable or unfavorable clinical outcome of disease, responsiveness or non-responsiveness to a given treatment or the like. Classification may be performed by use of an algorithm, in particular a discriminate function. A simple example of an algorithm is classification according to a first quantitative parameter, e.g. expression level of a nucleic acid of interest, being above or below a certain threshold value. Classification of a sample of a patient may be used to predict an outcome of disease or the risk of developing a disease. Instead of using the expression level of a single nucleic acid of interest, a combined score of several nucleic acids of interest of interest may be used. Further, additional data may be used in combination with the first quantitative parameter. Such additional data may be clinical data from the patient, such as sex, age, weight of the patient, disease grading etc.

A “discriminant function” is a function of a set of variables used to classify an object or event. A discriminant function thus allows classification of a patient, sample or event into a category or a plurality of categories according to data or parameters available from said patient, sample or event. Such classification is a standard instrument of statistical analysis well known to the skilled person. E.g. a patient may be classified as “high risk” or “low risk”, “in need of treatment” or “not in need of treatment” or other categories according to data obtained from said patient, sample or event. Classification is not limited to “high vs. low”, but may be performed into a plurality of categories, grading or the like. Examples for discriminant functions which allow a classification include, but are not limited to discriminant functions defined by support vector machines (SVM), k-nearest neighbors (kNN), (naive) Bayes models, or piecewise defined functions such as, for example, in subgroup discovery, in decision trees, in logical analysis of data (LAD) an the like.

The term “expression level” refers, e.g., to a determined level of expression of a nucleic acid of interest. The term “pattern of expression levels” refers to a determined level of expression com-pared either to a reference nucleic acid, e.g. from a control, or to a computed average expression value, e.g. in DNA-chip analyses. A pattern is not limited to the comparison of two genes but is also related to multiple comparisons of genes to reference genes or samples. A certain “pattern of expression levels” may also result and be deter-mined by comparison and measurement of several nucleic acids of interest disclosed hereafter and display the relative abundance of these transcripts to each other. Expression levels may also be assessed relative to expression in different tissues, patients versus healthy controls, etc.

A “reference pattern of expression levels”, within the meaning of the invention shall be understood as being any pattern of expression levels that can be used for the comparison to another pattern of expression levels. In a preferred embodiment of the invention, a reference pattern of expression levels is, e.g., an average pattern of expression levels observed in a group of healthy or diseased individuals, serving as a reference group.

In the context of the present invention a “sample” or a “biological sample” is a sample, which is derived from or has been in contact with a biological organism. Examples for biological samples are: cells, tissue, body fluids, biopsy specimens, blood, urine, saliva, sputum, plasma, serum, cell culture supernatant, and others.

A “probe” is a molecule or substance capable of specifically binding or interacting with a specific biological molecule. The term “primer”, “primer pair” or “probe”, shall have ordinary meaning of these terms which is known to the person skilled in the art of molecular biology. In a preferred embodiment of the invention “primer”, “primer pair” and “probes” refer to oligonucleotide or polynucleotide molecules with a sequence identical to, complementary too, homologues of, or homologous to regions of the target molecule or target sequence which is to be detected or quantified, such that the primer, primer pair or probe can specifically bind to the target molecule, e.g. target nucleic acid, RNA, DNA, cDNA, gene, transcript, peptide, polypeptide, or protein to be detected or quantified. As understood herein, a primer may in itself function as a probe. A “probe” as understood herein may also comprise e.g. a combination of primer pair and internal labeled probe, as is common in many commercially available qPCR methods.

A “gene” is a set of segments of nucleic acid that contains the information necessary to produce a functional RNA product in a controlled manner. A “gene product” is a biological molecule produced through transcription or expression of a gene, e.g. an mRNA or the translated protein.

A “miRNA” is a short, naturally occurring RNA molecule and shall have the ordinary meaning understood by a person skilled in the art. A “molecule derived from a miRNA” is a molecule which is chemically or enzymatically obtained from a miRNA template, such as cDNA.

The term “array” refers to an arrangement of addressable locations on a device, e.g. a chip device. The number of locations can range from several to at least hundreds or thousands. Each location represents an independent reaction site. Arrays include, but are not limited to nucleic acid arrays, protein arrays and antibody arrays. A “nucleic acid array” refers to an array containing nucleic acid probes, such as oligonucleotides, polynucleotides or larger portions of genes. The nucleic acid on the array is preferably single stranded. A “microarray” refers to a biochip or biological chip, i.e. an array of regions having a density of discrete regions with immobilized probes of at least about 100/cm².

A “PCR-based method” refers to methods comprising a polymerase chain reaction PCR. This is a method of exponentially amplifying nucleic acids, e.g. DNA or RNA by enzymatic replication in vitro using one, two or more primers. For RNA amplification, a reverse transcription may be used as a first step. PCR-based methods comprise kinetic or quantitative PCR (qPCR) which is particularly suited for the analysis of expression levels,). When it comes to the determination of expression levels, a PCR based method may for example be used to detect the presence of a given mRNA by (1) reverse transcription of the complete mRNA pool (the so called transcriptome) into cDNA with help of a reverse transcriptase enzyme, and (2) detecting the presence of a given cDNA with help of respective primers. This approach is commonly known as reverse transcriptase PCR (rtPCR). The term “PCR based method” comprises both end-point PCR applications as well as kinetic/real time PCR techniques applying special fluorophors or intercalating dyes which emit fluorescent signals as a function of amplified target and allow monitoring and quantification of the target. Quantification methods could be either absolute by external standard curves or relative to a comparative internal standard.

The term “next generation sequencing” or “high throughput sequencing” refers to high-throughput sequencing technologies that parallelize the sequencing process, producing thousands or millions of sequences at once. Examples include Massively Parallel Signature Sequencing (MPSS) Polony sequencing, 454 pyrosequencing, Illumina (Solexa) sequencing, SOLiD sequencing, Ion semiconductor sequencing, DNA nanoball sequencing, Helioscope™ single molecule sequencing, Single Molecule SMRT™ sequencing, Single Molecule real time (RNAP) sequencing, Nanopore DNA sequencing.

The term “marker” or “biomarker” refers to a biological molecule, e.g., a nucleic acid, peptide, protein, hormone, etc., whose presence or concentration can be detected and correlated with a known condition, such as a disease state, or with a clinical outcome, such as response to a treatment.

In its most general terms, the invention relates to a collection of miRNA markers useful for the diagnosis, prognosis and prediction of neuromyelitis optica, in particular to differentiate between NMO and MS (including CIS/RRMS).

The invention relates to a method for diagnosing neuromyelitis optica, predicting risk of developing neuromyelitis optica, or predicting an outcome of neuromyelitis optica in a patient suffering from or at risk of developing neuromyelitis optica, said method comprising the steps of:

a) determining in a sample from said patient, the expression level of at least one miRNA selected from the group consisting of the miRNA species hsa-miR-6131, hsa-miR-127-3p, hsa-miR-181a-2-3p, hsa-miR-6775-3p, hsa-miR-454-5p, hsa-miR-6735-3p, hsa-miR-23b-3p, hsa-miR-6840-5p, hsa-miR-4301, hsa-miR-6798-3p, hsa-miR-6513-3p, hsa-miR-28-5p, hsa-miR-181b-5p, hsa-miR-943, hsa-miR-6501-5p, hsa-miR-1287-5p, hsa-miR-3605-3p, hsa-miR-4448, hsa-miR-3127-3p, hsa-miR-942-5p, hsa-miR-6737-3p, hsa-miR-4755-3p, hsa-miR-3150a-5p, hsa-miR-6762-3p, hsa-miR-505-5p, hsa-let-7f-5p, hsa-miR-6819-3p, hsa-miR-127-3p, hsa-miR-223-5p, hsa-miR-3912-3p, hsa-miR-5094, hsa-miR-6818-3p, hsa-miR-1468-5p, hsa-miR-379-3p, hsa-miR-411-3p, hsa-miR-301b, hsa-miR-6505-3p, hsa-miR-671-3p, hsa-miR-3934-5p, hsa-miR-1304-5p, hsa-miR-4753-3p, hsa-miR-4775, hsa-miR-493-3p, hsa-miR-451b, hsa-miR-548ac, hsa-miR-4662a-5p, hsa-miR-548q, hsa-miR-409-5p, hsa-miR-1908-3p, hsa-miR-937-3p, hsa-miR-651-5p, hsa-miR-188-5p, hsa-miR-548b-5p, hsa-miR-4714-5p, hsa-miR-6735-3p, hsa-miR-4504, hsa-miR-4635, hsa-miR-548n, hsa-miR-3128, hsa-miR-421, hsa-miR-6783-5p, hsa-miR-3677-3p, hsa-miR-6737-3p, hsa-miR-486-3p, hsa-miR-7-5p, hsa-miR-548t-3p, hsa-miR-450b-5p, also listed in table 1;
b) comparing the pattern of expression level(s) determined in step a) with one or several reference pattern(s) of expression levels; and
c) diagnosing neuromyelitis optica, predicting risk of developing neuromyelitis optica, or predicting an outcome of neuromyelitis optica from the outcome of the comparison in step b).

Further the invention relates to a method of classifying a sample of a patient suffering from or at risk of developing neuromyelitis optica, said method comprising the steps of:

a) determining in a sample from said patient, the expression level of at least one miRNA selected from the group consisting of the miRNA species hsa-miR-6131, hsa-miR-127-3p, hsa-miR-181a-2-3p, hsa-miR-6775-3p, hsa-miR-454-5p, hsa-miR-6735-3p, hsa-miR-23b-3p, hsa-miR-6840-5p, hsa-miR-4301, hsa-miR-6798-3p, hsa-miR-6513-3p, hsa-miR-28-5p, hsa-miR-181b-5p, hsa-miR-943, hsa-miR-6501-5p, hsa-miR-1287-5p, hsa-miR-3605-3p, hsa-miR-4448, hsa-miR-3127-3p, hsa-miR-942-5p, hsa-miR-6737-3p, hsa-miR-4755-3p, hsa-miR-3150a-5p, hsa-miR-6762-3p, hsa-miR-505-5p, hsa-let-7f-5p, hsa-miR-6819-3p, hsa-miR-127-3p, hsa-miR-223-5p, hsa-miR-3912-3p, hsa-miR-5094, hsa-miR-6818-3p, hsa-miR-1468-5p, hsa-miR-379-3p, hsa-miR-411-3p, hsa-miR-301b, hsa-miR-6505-3p, hsa-miR-671-3p, hsa-miR-3934-5p, hsa-miR-1304-5p, hsa-miR-4753-3p, hsa-miR-4775, hsa-miR-493-3p, hsa-miR-451b, hsa-miR-548ac, hsa-miR-4662a-5p, hsa-miR-548q, hsa-miR-409-5p, hsa-miR-1908-3p, hsa-miR-937-3p, hsa-miR-651-5p, hsa-miR-188-5p, hsa-miR-548b-5p, hsa-miR-4714-5p, hsa-miR-6735-3p, hsa-miR-4504, hsa-miR-4635, hsa-miR-548n, hsa-miR-3128, hsa-miR-421, hsa-miR-6783-5p, hsa-miR-3677-3p, hsa-miR-6737-3p, hsa-miR-486-3p, hsa-miR-7-5p, hsa-miR-548t-3p, hsa-miR-450b-5p, also listed in table 1;
b) comparing the pattern of expression level(s) determined in step a) with one or several reference pattern(s) of expression levels; and;
c) classifying the sample of said patient from the outcome of the comparison in step b) into one of at least two classes indicative of a diagnosis of neuromyelitis optica, of predicting a risk of developing neuromyelitis optica, or of predicting an outcome of neuromyelitis optica.
Such classification can be indicative of a diagnosis of multiple sclerosis, of predicting a risk of developing multiple sclerosis, or of predicting an outcome of multiple sclerosis

Said classes may be healthy/diseased, low risk/high risk, low risk/high risk of developing disease or the like.

Preferably, the expression level of 2, 3, 4, 5, 6, 7, 8, 9, 10, 12, 15, or more miRNAs can be determined in said sample from said patient.

A reference pattern of expression levels may be obtained by determining in at least one healthy subject or at least one subject suffering from MS (including CIS/RRMS) the expression level of the at least one miRNA.

It is within the scope of the invention to assign a numerical value to an expression level of the at least one miRNA determined in step a).

It is further within the scope of the invention to apply an algorithm to perform step b) by applying an algorithm to obtain a normalized expression level relative to a reference pattern of expression level(s).

It is within the scope of the invention to apply an algorithm to the numerical value of the expression level of the at least one miRNA determined in step a) to obtain a disease score to allow classification of the sample or diagnosis, prognosis or prediction of the risk of developing neuromyelitis optica, or prediction of an outcome of neuromyelitis optica. A non-limiting example of such an algorithm is to compare the numerical value of the expression level against a threshold value in order to classify the result into one of two categories, such as high risk/low risk, diseased/healthy or the like. A further non-limiting example of such an algorithm is to combine a plurality of numerical values of expression levels, e.g. by summation, to obtain a combined score. Individual summands may be normalized or weighted by multiplication with factors or numerical values representing the expression level of a miRNA, numerical values representing clinical data, or other factors.

It is within the scope of the invention to apply a discriminant function to classify a result, diagnose disease, predict an outcome or a risk.

According to an aspect of the invention, the sample is selected from the group consisting of blood sample, serum sample, and plasma sample.

According to a further aspect of the invention the sample is a blood sample.

According to an aspect of the invention the methods of the invention comprise in step a) determining the expression level of at least one miRNA selected from the group consisting of the miRNA species hsa-miR-6131, hsa-miR-5094, hsa-miR-223-5p, hsa-miR-4753-3p, hsa-miR-6775-3p, hsa-miR-548b-5p, hsa-miR-3912-3p, hsa-miR-4714-5p, hsa-miR-6798-3p, hsa-miR-6501-5p, hsa-miR-454-5p, hsa-miR-6735-3p, hsa-miR-4504, hsa-miR-4301, hsa-miR-4635, hsa-miR-548n, hsa-miR-3128, hsa-miR-421, hsa-miR-1908-3p, hsa-miR-943, hsa-miR-6783-5p, hsa-miR-3677-3p, hsa-miR-3127-3p, hsa-miR-6737-3p, hsa-miR-486-3p, hsa-miR-7-5p, hsa-miR-548t-3p, hsa-miR-450b-5p, hsa-miR-486-3p, hsa-miR-505-5p as listed in table 2. According to this aspect the method may be used in particular to determine whether said patient is suffering from or at risk of developing neuromyelitis optica or not.

According to an aspect of the invention the methods of the invention comprise in step a) determining the expression level of the miRNA: hsa-miR-6131.

According to an aspect of the invention the methods of the invention comprise determining from the outcome of step b) and/or c) whether a patient is suffering from or at risk of developing neuromyelitis optica versus multiple sclerosis.

According to this aspect of the invention (comprising determining whether a patient is suffering from or at risk of developing NMO versus MS), the methods of the invention comprise in step a) determining the expression level of the at least one miRNA selected from the group consisting of the miRNA species hsa-miR-6131 and hsa-miR-127-3p.

According to this aspect of the invention (comprising determining whether a patient is suffering from or at risk of developing NMO versus MS), the methods of the invention comprise in step a) determining the expression level of of at least one miRNA selected from the group consisting of the miRNA species hsa-miR-6131, hsa-miR-127-3p hsa-miR-181a-2-3p, hsa-miR-6775-3p, hsa-miR-454-5p, hsa-miR-6735-3p, hsa-miR-23b-3p, hsa-miR-6840-5p, hsa-miR-4301, hsa-miR-6798-3p, hsa-miR-6513-3p, hsa-miR-28-5p, hsa-miR-181b-5p, hsa-miR-181b-5p, hsa-miR-943, hsa-miR-3127-5p, hsa-miR-6501-5p, hsa-miR-1287-5p, hsa-miR-3605-3p, hsa-miR-4448, hsa-miR-3127-3p, hsa-miR-942-5p, hsa-miR-6737-3p, hsa-miR-4755-3p, hsa-miR-3150a-5p, hsa-miR-6762-3p, hsa-miR-505-5p, hsa-let-7f-5p, hsa-miR-6819-3p, as listed in table 3.

According to this aspect of the invention (comprising determining whether a patient is suffering from or at risk of developing NMO versus MS), the methods of the invention comprise in step a) determining the expression level of of at least one miRNA selected from the group consisting of the miRNA species hsa-miR-6131, hsa-miR-127-3p, hsa-miR-127-3p, hsa-miR-223-5p, hsa-miR-6737-3p, hsa-miR-3912-3p, hsa-miR-5094, hsa-miR-6735-3p, hsa-miR-6818-3p, hsa-miR-1468-5p, hsa-miR-379-3p, hsa-miR-411-3p, hsa-miR-301b, hsa-miR-6505-3p, hsa-miR-671-3p, hsa-miR-3934-5p, hsa-miR-1304-5p, hsa-miR-4753-3p, hsa-miR-4775, hsa-miR-4755-3p, hsa-miR-493-3p, hsa-miR-451b, hsa-miR-411-5p, hsa-miR-548ac, hsa-miR-4662a-5p, hsa-miR-548q, hsa-miR-409-5p, hsa-miR-1908-3p, hsa-miR-937-3p, hsa-miR-651-5p, hsa-miR-188-5p, as listed in table 4.

According to an aspect of the invention, the methods of the invention comprise in step a) determining the expression level of at least one miRNA selected from the group consisting of the miRNA species hsa-miR-6131 and hsa-miR-127-3p, and at least one further miRNA selected from the group consisting of the miRNA species listed in any of the tables 1, 2, 3, and 4. According to this aspect, preferably, the expression level of 2, 3, 4, 5, 6, 7, 8, 9, 10, 12, 15, or more further miRNAs can be determined in said sample from said patient.

According to an aspect of the invention, preferably the markers or combinations of miRNA markers to be used comprise or consist of the following marker combinations:
hsa-miR-6131;
hsa-miR-127-3p;
hsa-miR-6131, hsa-miR-127-3p;
hsa-miR-6131, hsa-miR-5094;
hsa-miR-6131, hsa-miR-5094, hsa-miR-223-5p,
hsa-miR-6131, hsa-miR-5094, hsa-miR-223-5p, hsa-miR-4753-3p,
hsa-miR-6131, hsa-miR-5094, hsa-miR-223-5p, hsa-miR-4753-3p, hsa-miR-6775-3p,
hsa-miR-6131, hsa-miR-5094, hsa-miR-223-5p, hsa-miR-4753-3p, hsa-miR-6775-3p, hsa-miR-548b-5p,
hsa-miR-223-5p, hsa-miR-4753-3p, hsa-miR-6775-3p,
hsa-miR-223-5p, hsa-miR-4753-3p, hsa-miR-6775-3p, hsa-miR-548b-5p,
hsa-miR-223-5p, hsa-miR-4753-3p, hsa-miR-6775-3p, hsa-miR-548b-5p, hsa-miR-3912-3p,
hsa-miR-223-5p, hsa-miR-4753-3p, hsa-miR-6775-3p, hsa-miR-548b-5p, hsa-miR-3912-3p, hsa-miR-4714-5p,
hsa-miR-223-5p, hsa-miR-4753-3p, hsa-miR-6775-3p, hsa-miR-548b-5p, hsa-miR-3912-3p, hsa-miR-4714-5p, hsa-miR-6798-3p,
hsa-miR-223-5p, hsa-miR-4753-3p, hsa-miR-6775-3p, hsa-miR-548b-5p, hsa-miR-3912-3p, hsa-miR-4714-5p, hsa-miR-6798-3p, hsa-miR-6501-5p,
hsa-miR-223-5p, hsa-miR-4753-3p, hsa-miR-6775-3p, hsa-miR-548b-5p, hsa-miR-3912-3p, hsa-miR-4714-5p, hsa-miR-6798-3p, hsa-miR-6501-5p, hsa-miR-454-5p,
hsa-miR-6131, hsa-miR-127-3p, hsa-miR-181a-2-3p,
hsa-miR-6131, hsa-miR-127-3p, hsa-miR-181a-2-3p, hsa-miR-6775-3p,
hsa-miR-6131, hsa-miR-127-3p, hsa-miR-181a-2-3p, hsa-miR-6775-3p, hsa-miR-454-5p,
hsa-miR-181a-2-3p, hsa-miR-6775-3p,
hsa-miR-181a-2-3p, hsa-miR-6775-3p, hsa-miR-454-5p,
hsa-miR-181a-2-3p, hsa-miR-6775-3p, hsa-miR-454-5p, hsa-miR-6735-3p, hsa-miR-23b-3p,
hsa-miR-181a-2-3p, hsa-miR-6775-3p, hsa-miR-454-5p, hsa-miR-6735-3p, hsa-miR-23b-3p, hsa-miR-6840-5p,
hsa-miR-181a-2-3p, hsa-miR-6775-3p, hsa-miR-454-5p, hsa-miR-6735-3p, hsa-miR-23b-3p, hsa-miR-6840-5p, hsa-miR-4301,
hsa-miR-181a-2-3p, hsa-miR-6775-3p, hsa-miR-454-5p, hsa-miR-6735-3p, hsa-miR-23b-3p, hsa-miR-6840-5p, hsa-miR-4301, hsa-miR-6798-3p,
hsa-miR-181a-2-3p, hsa-miR-6775-3p, hsa-miR-454-5p, hsa-miR-6735-3p, hsa-miR-23b-3p, hsa-miR-6840-5p, hsa-miR-4301, hsa-miR-6798-3p, hsa-miR-6513-3p,
hsa-miR-6131, hsa-miR-127-3p, hsa-miR-223-5p,
hsa-miR-6131, hsa-miR-127-3p, hsa-miR-223-5p, hsa-miR-6737-3p,
hsa-miR-6131, hsa-miR-127-3p, hsa-miR-223-5p, hsa-miR-6737-3p, hsa-miR-3912-3p,
hsa-miR-6131, hsa-miR-127-3p, hsa-miR-223-5p, hsa-miR-6737-3p, hsa-miR-3912-3p, hsa-miR-5094,
hsa-miR-223-5p, hsa-miR-6737-3p, hsa-miR-3912-3p,
hsa-miR-223-5p, hsa-miR-6737-3p, hsa-miR-3912-3p, hsa-miR-5094,
hsa-miR-223-5p, hsa-miR-6737-3p, hsa-miR-3912-3p, hsa-miR-5094, hsa-miR-6735-3p,
hsa-miR-223-5p, hsa-miR-6737-3p, hsa-miR-3912-3p, hsa-miR-5094, hsa-miR-6735-3p, hsa-miR-6818-3p,
hsa-miR-223-5p, hsa-miR-6737-3p, hsa-miR-3912-3p, hsa-miR-5094, hsa-miR-6735-3p, hsa-miR-6818-3p, hsa-miR-1468-5p, and
hsa-miR-223-5p, hsa-miR-6737-3p, hsa-miR-3912-3p, hsa-miR-5094, hsa-miR-6735-3p, hsa-miR-6818-3p, hsa-miR-1468-5p, hsa-miR-379-3p.

According to an aspect of the invention, the determination of the expression level in step (a) is obtained by use of a method selected from the group consisting of a Sequencing-based method, an array based method and a PCR based method.

The invention further relates to a kit for diagnosing neuromyelitis optica, predicting risk of developing neuromyelitis optica, or predicting an outcome of neuromyelitis optica in a patient suffering from or at risk of developing neuromyelitis optica, said kit comprising
means for determining in said sample from said patient, an expression level of at least one miRNA selected from the group consisting of hsa-miR-6131, hsa-miR-127-3p, hsa-miR-181a-2-3p, hsa-miR-6775-3p, hsa-miR-454-5p, hsa-miR-6735-3p, hsa-miR-23b-3p, hsa-miR-6840-5p, hsa-miR-4301, hsa-miR-6798-3p, hsa-miR-6513-3p, hsa-miR-28-5p, hsa-miR-181b-5p, hsa-miR-943, hsa-miR-6501-5p, hsa-miR-1287-5p, hsa-miR-3605-3p, hsa-miR-4448, hsa-miR-3127-3p, hsa-miR-942-5p, hsa-miR-6737-3p, hsa-miR-4755-3p, hsa-miR-3150a-5p, hsa-miR-6762-3p, hsa-miR-505-5p, hsa-let-7f-5p, hsa-miR-6819-3p, hsa-miR-127-3p, hsa-miR-223-5p, hsa-miR-3912-3p, hsa-miR-5094, hsa-miR-6818-3p, hsa-miR-1468-5p, hsa-miR-379-3p, hsa-miR-411-3p, hsa-miR-301b, hsa-miR-6505-3p, hsa-miR-671-3p, hsa-miR-3934-5p, hsa-miR-1304-5p, hsa-miR-4753-3p, hsa-miR-4775, hsa-miR-493-3p, hsa-miR-451b, hsa-miR-548ac, hsa-miR-4662a-5p, hsa-miR-548q, hsa-miR-409-5p, hsa-miR-1908-3p, hsa-miR-937-3p, hsa-miR-651-5p, hsa-miR-188-5p, hsa-miR-548b-5p, hsa-miR-4714-5p, hsa-miR-6735-3p, hsa-miR-4504, hsa-miR-4635, hsa-miR-548n, hsa-miR-3128, hsa-miR-421, hsa-miR-6783-5p, hsa-miR-3677-3p, hsa-miR-6737-3p, hsa-miR-486-3p, hsa-miR-7-5p, hsa-miR-548t-3p, hsa-miR-450b-5p, also listed in table 1, and
at least one reference pattern of expression levels for comparing with the expression level of the at least one miRNA from said sample.

The means for determining the expression level of said at least one miRNA may comprise an oligonucleotide probe for detecting or amplifying said at least one miRNA, means for determining the expression level based on an array-based method, a PCR based method, a sequencing based method or any other suitable means for determining the expression level.

The reference expression level pattern may be supplied as numeric information, in particular as computer-encoded information on any suitable information carrier.

The invention further relates to computer program product for diagnosing neuromyelitis optica, predicting risk of developing neuromyelitis optica, or predicting an outcome of neuromyelitis optica in a patient suffering from or at risk of developing neuromyelitis optica, comprising
means for receiving data representing an expression level of at least one miRNA in a patient sample selected from the group consisting of hsa-miR-6131, hsa-miR-127-3p, hsa-miR-181a-2-3p, hsa-miR-6775-3p, hsa-miR-454-5p, hsa-miR-6735-3p, hsa-miR-23b-3p, hsa-miR-6840-5p, hsa-miR-4301, hsa-miR-6798-3p, hsa-miR-6513-3p, hsa-miR-28-5p, hsa-miR-181b-5p, hsa-miR-943, hsa-miR-6501-5p, hsa-miR-1287-5p, hsa-miR-3605-3p, hsa-miR-4448, hsa-miR-3127-3p, hsa-miR-942-5p, hsa-miR-6737-3p, hsa-miR-4755-3p, hsa-miR-3150a-5p, hsa-miR-6762-3p, hsa-miR-505-5p, hsa-let-7f-5p, hsa-miR-6819-3p, hsa-miR-127-3p, hsa-miR-223-5p, hsa-miR-3912-3p, hsa-miR-5094, hsa-miR-6818-3p, hsa-miR-1468-5p, hsa-miR-379-3p, hsa-miR-411-3p, hsa-miR-301b, hsa-miR-6505-3p, hsa-miR-671-3p, hsa-miR-3934-5p, hsa-miR-1304-5p, hsa-miR-4753-3p, hsa-miR-4775, hsa-miR-493-3p, hsa-miR-451b, hsa-miR-548ac, hsa-miR-4662a-5p, hsa-miR-548q, hsa-miR-409-5p, hsa-miR-1908-3p, hsa-miR-937-3p, hsa-miR-651-5p, hsa-miR-188-5p, hsa-miR-548b-5p, hsa-miR-4714-5p, hsa-miR-6735-3p, hsa-miR-4504, hsa-miR-4635, hsa-miR-548n, hsa-miR-3128, hsa-miR-421, hsa-miR-6783-5p, hsa-miR-3677-3p, hsa-miR-6737-3p, hsa-miR-486-3p, hsa-miR-7-5p, hsa-miR-548t-3p, hsa-miR-450b-5p, also listed in table 1,
means for receiving data representing at least one reference pattern of expression levels for comparing with the expression level of the at least one miRNA from said sample,
means for comparing said data representing the expression level of the at least one miRNA in a patient sample, and
means for determining a diagnosis of neuromyelitis optica, a prediction of a risk of developing neuromyelitis optica, or a prediction of an outcome of neuromyelitis optica from the outcome of the comparison in step b).

The computer program product may be provided on a storable electronic medium, such as a solid state memory, disk, CD or other. The computer program product may be stored on non-transitory computer-readable medium adapted to operate on one or more computers, the computer-readable medium comprising:

storage media containing It may be stored locally on a computer. It may be implemented as network-based program or application, including a web- or internet-based application. It may be implemented in a diagnostic device, such as an analyzer instrument. It may be operably connected to a device for outputting information, such as a display, printer or the like.

### Examples

Additional details, features, characteristics and advantages of the object of the invention are further disclosed in the following description and figures of the respective examples, which, in an exemplary fashion, show preferred embodiments of the present invention. However, these examples should by no means be understood as to limit the scope of the invention.

The invention relates to methods of differential diagnosis of neuromyelitis optica (NMO) vs. multiple sclerosis (MS) using miRNA biomarkers.

Diagnosis of multiple sclerosis (MS) can be challenging in patients with atypical presentations and during a first neurological deficit possibly related to inflammatory demyelination. In particular, it is difficult to differentiate NMO and MS, including CIS/RRMS, which often presents the earliest stage of disease. However, it would be particularly desirable to have a reliable diagnostic test for this differentiation. Towards the identification of biomarkers for diagnosis of NMO, a comprehensive analysis of miRNA expression patterns in whole blood samples from treatment-naive patients with confirmed NMO, a clinically isolated syndrome (CIS) or relapsing-remitting MS (RRMS) and matched controls by using Next Generation Sequencing, microarray analysis, and qRT-PCR was obtained. In patients with NMO, significantly deregulated miRNAs were identified. These miRNAs could potentially serve as future diagnostic biomarkers.

### Patients and Sample Preparation

About 5 ml of blood was collected in PAXgene Blood RNA tubes (Becton Dickinson, Heidelberg, Germany) from patients/controls.

Total RNA including miRNA was isolated using the PAXgene Blood miRNA Kit (Qiagen) following the manufacturers recommendations. Isolated RNA was stored at −80° C. RNA integrity was analyzed using Bioanalyzer 2100 (Agilent) and concentration and purity was measured using NanoDrop 2000 (thermo Scientific).

### NGS Screening

Initially a high-throughput screening of 38 samples from X patients with NMO, Y1 CIS/RRMS patients in a first cohort and Y2 CIS/RRMS patients in a second cohort and Z controls was performed. Altogether ca. 3000 miRNA markers were analyzed. Table 1 shows mRNI markers that were found to be significantly deregulated in patients with NMO. The second column refers to the SEQ ID NO of the Sequence Listing.

TABLE 1

| | SEQ | | |
|---|---|---|---|
| No. | ID NO | miRNA | SEQUENZ |
| 1 | 1 | hsa-miR- | ACCACUGACCGUUGACUGUACC |
| | | 181a-2-3p | |
| 2 | 2 | hsa-miR- | AGGCCCUGUCCUCUGCCCCAG |
| | | 6775-3p | |
| 3 | 3 | hsa-miR- | ACCCUAUCAAUAUUGUCUCUGC |
| | | 454-5p | |
| 4 | 4 | hsa-miR- | AGGCCUGUGGCUCCUCCCUCAG |
| | | 6735-3p | |
| 5 | 5 | hsa-miR- | AUCACAUUGCCAGGGAUUACC |
| | | 23b-3p | |
| 6 | 6 | hsa-miR- | GGCUGGUCAGAUGGGAGUG |
| | | 6131 | |
| 7 | 7 | hsa-miR- | ACCCCCGGGCAAAGACCUGCAGAU |
| | | 6840-5p | |
| 8 | 8 | hsa-miR- | UCCCACUACUUCACUUGUGA |
| | | 4301 | |
| 9 | 9 | hsa-miR- | CUACCCCCCAUCCCCCUGUAG |
| | | 6798-3p | |
| 10 | 10 | hsa-miR- | UCAAGUGUCAUCUGUCCCUAG |
| | | 6513-3p | |
| 11 | 11 | hsa-miR- | AAGGAGCUCACAGUCUAUUGAG |
| | | 28-5p | |
| 12 | 12 | hsa-miR- | AACAUUCAUUGCUGUCGGUGGGU |
| | | 181b-5p | |
| 14 | 13 | hsa-miR- | CUGACUGUUGCCGUCCUCCAG |
| | | 943 | |
| 16 | 14 | hsa-miR- | AGUUGCCAGGGCUGCCUUUGGU |
| | | 6501-5p | |
| 17 | 15 | hsa-miR- | UGCUGGAUCAGUGGUUCGAGUC |
| | | 1287-5p | |
| 18 | 16 | hsa-miR- | CCUCCGUGUUACCUGUCCUCUAG |
| | | 3605-3p | |
| 19 | 17 | hsa-miR- | GGCUCCUUGGUCUAGGGGUA |
| | | 4448 | |
| 20 | 18 | hsa-miR- | UCCCCUUCUGCAGGCCUGCUGG |
| | | 3127-3p | |
| 21 | 19 | hsa-miR- | UCUUCUCUGUUUUGGCCAUGUG |
| | | 942-5p | |
| 22 | 20 | hsa-miR- | UCUGUGCUUCACCCCUACCCAG |
| | | 6737-3p | |
| 23 | 21 | hsa-miR- | AGCCAGGCUCUGAAGGGAAAGU |
| | | 4755-3p | |
| 24 | 22 | hsa-miR- | CAACCUCGACGAUCUCCUCAGC |
| | | 3150a-5p | |
| 25 | 23 | hsa-miR- | UGGCUGCUUCCCUUGGUCUCCAG |
| | | 6762-3p | |
| 26 | 24 | hsa-miR- | GGGAGCCAGGAAGUAUUGAUGU |
| | | 505-5p | |
| 27 | 25 | hsa-let- | UGAGGUAGUAGAUUGUAUAGUU |
| | | 71-5p | |
| 28 | 26 | hsa-miR- | AAGCCUCUGUCCCCACCCCAG |
| | | 6819-3p | |
| 29 | 27 | hsa-miR- | UCGGAUCCGUCUGAGCUUGGCU |
| | | 127-3p | |
| 31 | 28 | hsa-miR- | CGUGUAUUUGACAAGCUGAGUU |
| | | 223-5p | |
| 33 | 29 | hsa-miR- | UAACGCAUAAUAUGGACAUGU |
| | | 3912-3p | |
| 34 | 30 | hsa-miR- | AAUCAGUGAAUGCCUUGAACCU |
| | | 5094 | |
| 37 | 31 | hsa-miR- | UUGUCUCUUGUUCCUCACACAG |
| | | 6818-3p | |
| 38 | 32 | hsa-miR- | CUCCGUUUGCCUGUUUCGCUG |
| | | 1468-5p | |
| 39 | 33 | hsa-miR- | UAUGUAACAUGGUCCACUAACU |
| | | 379-3p | |
| 40 | 34 | hsa-miR- | UAUGUAACACGGUCCACUAACC |
| | | 411-3p | |
| 41 | 35 | hsa-miR- | CAGUGCAAUGAUAUUGUCAAAGC |
| | | 301b | |
| 42 | 36 | hsa-miR- | UGACUUCUACCUCUUCCAAAG |
| | | 6505-3p | |
| 43 | 37 | hsa-miR- | UCCGGUUCUCAGGGCUCCACC |
| | | 671-3p | |
| 44 | 38 | hsa-miR- | UCAGGUGUGGAAACUGAGGCAG |
| | | 3934-5p | |
| 45 | 39 | hsa-miR- | UUUGAGGCUACAGUGAGAUGUG |
| | | 1304-5p | |
| 46 | 40 | hsa-miR- | UUCUCUUUCUUUAGCCUUGUGU |
| | | 4753-3p | |
| 48 | 41 | hsa-miR- | UUAAUUUUUUGUUUCGGUCACU |
| | | 4775 | |
| 50 | 42 | hsa-miR- | UGAAGGUCUACUGUGUGCCAGG |
| | | 493-3p | |
| 51 | 43 | hsa-miR- | UAGCAAGAGAACCAUUACCAUU |
| | | 451b | |
| 53 | 44 | hsa-miR- | CAAAAACCGGCAAUUACUUUUG |
| | | 548ac | |
| 54 | 45 | hsa-miR- | UUAGCCAAUUGUCCAUCUUUAG |
| | | 4662a-5p | |
| 55 | 46 | hsa-miR- | GCUGGUGCAAAAGUAAUGGCGG |
| | | 548q | |
| 56 | 47 | hsa-miR- | AGGUUACCCGAGCAACUUUGCAU |
| | | 409-5p | |
| 57 | 48 | hsa-miR- | CCGGCCGCCGGCUCCGCCCCG |
| | | 1908-3p | |
| 58 | 49 | hsa-miR- | AUCCGCGCUCUGACUCUCUGCC |
| | | 937-3p | |
| 59 | 50 | hsa-miR- | UUUAGGAUAAGCUUGACUUUUG |
| | | 651-5p | |
| 60 | 51 | hsa-miR- | CAUCCCUUGCAUGGUGGAGGG |
| | | 188-5p | |
| 66 | 52 | hsa-miR- | AAAAGUAAUUGUGGUUUUGGCC |
| | | 548b-5p | |
| 68 | 53 | hsa-miR- | AACUCUGACCCCUUAGGUUGAU |
| | | 4714-5p | |
| 72 | 54 | hsa-miR- | AGGCCUGUGGCUCCUCCCUCAG |
| | | 6735-3p | |
| 73 | 55 | hsa-miR- | UGUGACAAUAGAGAUGAACAUG |
| | | 4504 | |
| 75 | 56 | hsa-miR- | UCUUGAAGUCAGAACCCGCAA |
| | | 4635 | |
| 76 | 57 | hsa-miR- | CAAAAGUAAUUGUGGAUUUUGU |
| | | 548n | |
| 77 | 58 | hsa-miR- | UCUGGCAAGUAAAAAACUCUCAU |
| | | 3128 | |
| 78 | 59 | hsa-miR- | AUCAACAGACAUUAAUUGGGCGC |
| | | 421 | |
| 81 | 60 | hsa-miR- | UAGGGGAAAAGUCCUGAUCCGG |
| | | 6783-5p | |
| 82 | 61 | hsa-miR- | CUCGUGGGCUCUGGCCACGGCC |
| | | 3677-3p | |
| 84 | 62 | hsa-miR- | UCUGUGCUUCACCCCUACCCAG |
| | | 6737-3p | |
| 85 | 63 | hsa-miR- | CGGGGCAGCUCAGUACAGGAU |
| | | 486-3p | |
| 86 | 64 | hsa-miR- | UGGAAGACUAGUGAUUUUGUUGU |
| | | 7-5p | |
| 87 | 65 | hsa-miR- | AAAAACCACAAUUACUUUUGCACCA |
| | | 548t-3p | |
| 88 | 66 | hsa-miR- | UUUUGCAAUAUGUUCCUGAAUA |
| | | 450b-5p | |

The TruSeq Small RNA sample preparation Kit (Illumina) was used to generate multiplexed sequencing libraries, which were afterwards sequenced on a HiSeq2000 System (Illumina) using the 50 bp fragment sequencing protocol. Resulting sequencing reads were demultiplexed using the CASAVA 1.8 software package (Illumina) and quality checked using FastQC tools (Babraham Inst.). A primary mapping analysis using the miRDeep2-pipeline was conducted to ensure that a significant proportion of miRNAs have been sequenced.

On average, 1.5-2 million high quality sequencing reads per sample were obtained (at a total of 95 million reads) of which up to 70% contained miRNA information. The raw illumina reads were first preprocessed by cutting the 3′ adapter sequence. This was done by the program fastx_clipper from the FASTX-Toolkit. Reads shorter than 18 nucleotides after clipping were removed. The remaining reads were collapsed, i.e. after this step only unique reads and their frequency per sample was obtained. This step reduces the time for mapping the reads enormously. For the remaining steps, the miRDeep2 pipeline was used. These steps consist of mapping the reads against the genome (hg19), mapping the reads against miRNA precursor sequences from mirbase release v18, summarizing the counts for the samples, and prediction of novel miRNAs.

Tables 2, 3, and 4 show markers that were found to be significantly deregulated in patients with NMO vs. controls or two different cohorts of controls respectively. In tables 2, 3, and 4:
median g1 relates to the median number of reads in controls (table 2) or CIS/RRMS patients (tables 3 and 4);
median g2 relates to the median number of reads of NMO patients;
qmedian is the ratio of median g2/median g2, ttest_rawp indicates significance using the raw p value of a t test;
AUC indicates are under curve of a receiver/operator curve (ROC).

Table 2 shows markers that were found to be significantly deregulated in patients with NMO vs. controls.

TABLE 2
15_dmat_control_vs_nmo

| miRNA | median g1 | median g2 | qmedian | ttest_rawp | AUC |
|---|---|---|---|---|---|
| hsa-miR-6131 | 21.973684 | 1.004049 | 21.885081 | 0.000006 | 0.694215 |
| hsa-miR-5094 | 7.612348 | 1.004049 | 7.581653 | 0.000085 | 0.731405 |
| hsa-miR-223-5p | 6.586032 | 1.149798 | 5.727993 | 0.000187 | 0.651860 |
| hsa-miR-4753-3p | 1.004049 | 1.004049 | 1.000000 | 0.000291 | 0.664256 |
| hsa-miR-6775-3p | 4.574899 | 112.919028 | 0.040515 | 0.000553 | 0.128099 |
| hsa-miR-548b-5p | 1.004049 | 1.004049 | 1.000000 | 0.000632 | 0.644628 |
| hsa-miR-3912-3p | 1.004049 | 1.004049 | 1.000000 | 0.000767 | 0.652893 |
| hsa-miR-4714-5p | 1.004049 | 1.004049 | 1.000000 | 0.000931 | 0.669421 |
| hsa-miR-6798-3p | 1.004049 | 77.686235 | 0.012924 | 0.001659 | 0.120868 |
| hsa-miR-6501-5p | 20.937247 | 113.639676 | 0.184242 | 0.001671 | 0.140496 |
| hsa-miR-454-5p | 115.068826 | 29.161943 | 3.945856 | 0.001697 | 0.815083 |
| hsa-miR-6735-3p | 6.733806 | 69.421053 | 0.096999 | 0.002101 | 0.178719 |
| hsa-miR-4504 | 1.004049 | 1.004049 | 1.000000 | 0.002312 | 0.659091 |
| hsa-miR-4301 | 3.292510 | 73.425101 | 0.044842 | 0.002369 | 0.179752 |
| hsa-miR-4635 | 1.004049 | 2.635628 | 0.380952 | 0.002947 | 0.504132 |
| hsa-miR-548n | 1.004049 | 1.004049 | 1.000000 | 0.003074 | 0.644628 |
| hsa-miR-3128 | 1.004049 | 1.004049 | 1.000000 | 0.003217 | 0.654959 |
| hsa-miR-421 | 106.956478 | 30.467611 | 3.510498 | 0.004154 | 0.782025 |
| hsa-miR-1908-3p | 1.004049 | 73.425101 | 0.013674 | 0.005294 | 0.203512 |
| hsa-miR-943 | 4.585020 | 100.493927 | 0.045625 | 0.005299 | 0.185950 |
| hsa-miR-6783-5p | 1.004049 | 31.032389 | 0.032355 | 0.006319 | 0.149793 |
| hsa-miR-3677-3p | 1.004049 | 1.004049 | 1.000000 | 0.006337 | 0.640496 |
| hsa-miR-3127-3p | 48.436235 | 138.866397 | 0.348797 | 0.006360 | 0.185950 |
| hsa-miR-6737-3p | 1.004049 | 1.149798 | 0.873239 | 0.006571 | 0.529959 |
| hsa-miR-486-3p | 89.091093 | 40.732794 | 2.187208 | 0.006585 | 0.707645 |
| hsa-miR-7-5p | 31.491903 | 2.635628 | 11.948541 | 0.006675 | 0.694215 |
| hsa-miR-548t-3p | 1.004049 | 1.004049 | 1.000000 | 0.006830 | 0.591942 |
| hsa-miR-450b-5p | 1.004049 | 1.004049 | 1.000000 | 0.007420 | 0.588843 |
| hsa-miR-486-3p | 86.319838 | 40.732794 | 2.119173 | 0.007678 | 0.705579 |
| hsa-miR-505-5p | 9.298583 | 141.955466 | 0.065504 | 0.007847 | 0.188017 |

Preferred combinations of markers to be used in the methods, kits or computer program products of the invention comprise or consist of the first 2, 3, 4, 5, 6, 7, 8, 9, 10, 11, or 12 markers listed in table 2.

Table 3 shows markers that were found to be significantly deregulated in patients with NMO vs. a first cohort of patients with confirmed MS in the CIS or RRMS form.

TABLE 3
dmat_cis + rrms(1)_nmo

| miRNA | median g1 | median g2 | qmedian | ttest_rawp | AUC |
|---|---|---|---|---|---|
| hsa-miR-181a-2-3p | 157.663968 | 78.485830 | 2.008821 | 0.000008 | 0.933333 |
| hsa-miR-6775-3p | 1.004049 | 112.919028 | 0.008892 | 0.000076 | 0.042424 |
| hsa-miR-454-5p | 118.975709 | 29.161943 | 4.079828 | 0.000090 | 0.939394 |
| hsa-miR-6735-3p | 1.004049 | 69.421053 | 0.014463 | 0.000153 | 0.063636 |
| hsa-miR-23b-3p | 142.174089 | 60.062753 | 2.367092 | 0.000329 | 0.906061 |
| hsa-miR-6131 | 70.305668 | 1.004049 | 70.022177 | 0.000337 | 0.854545 |
| hsa-miR-6840-5p | 1.004049 | 86.064777 | 0.011666 | 0.000459 | 0.115152 |
| hsa-miR-4301 | 1.004049 | 73.425101 | 0.013674 | 0.000493 | 0.096970 |
| hsa-miR-6798-3p | 1.004049 | 77.686235 | 0.012924 | 0.000531 | 0.045455 |
| hsa-miR-6513-3p | 137.882591 | 45.518219 | 3.029174 | 0.000607 | 0.854545 |
| hsa-miR-28-5p | 147.929150 | 60.550607 | 2.443066 | 0.000737 | 0.896970 |
| hsa-miR-181b-5p | 145.615385 | 75.137652 | 1.937982 | 0.001071 | 0.854545 |
| hsa-miR-181b-5p | 143.315789 | 74.809717 | 1.915738 | 0.001095 | 0.854545 |
| hsa-miR-943 | 1.004049 | 100.493927 | 0.009991 | 0.001541 | 0.121212 |
| hsa-miR-3127-5p | 1.004049 | 91.876518 | 0.010928 | 0.001584 | 0.136364 |
| hsa-miR-6501-5p | 20.684211 | 113.639676 | 0.182016 | 0.001701 | 0.157576 |
| hsa-miR-1287-5p | 1.004049 | 86.064777 | 0.011666 | 0.001732 | 0.127273 |
| hsa-miR-3605-3p | 109.680162 | 73.732794 | 1.487536 | 0.001816 | 0.872727 |
| hsa-miR-4448 | 1.004049 | 102.761134 | 0.009771 | 0.001844 | 0.130303 |
| hsa-miR-3127-3p | 50.874494 | 138.866397 | 0.366356 | 0.002234 | 0.121212 |
| hsa-miR-942-5p | 152.285425 | 91.688259 | 1.660904 | 0.002414 | 0.833333 |
| hsa-miR-6737-3p | 28.439271 | 1.149798 | 24.734155 | 0.002531 | 0.763636 |
| hsa-miR-4755-3p | 1.004049 | 56.674089 | 0.017716 | 0.002783 | 0.124242 |
| hsa-miR-3150a-5p | 1.004049 | 56.674089 | 0.017716 | 0.002891 | 0.106061 |
| hsa-miR-6762-3p | 1.004049 | 56.674089 | 0.017716 | 0.002999 | 0.090909 |
| hsa-miR-505-5p | 16.253036 | 141.955466 | 0.114494 | 0.003054 | 0.178788 |
| hsa-let-7f-5p | 101.785425 | 43.348178 | 2.348090 | 0.003480 | 0.833333 |
| hsa-miR-6819-3p | 1.004049 | 56.342105 | 0.017821 | 0.003484 | 0.093939 |
| hsa-miR-127-3p | 99.477733 | 53.202429 | 1.869797 | 0.003495 | 0.800000 |

Preferred combinations of markers to be used in the methods, kits or computer program products of the invention comprise or consist of the first 2, 3, 4, 5, 6, 7, 8, 9, 10, 11, or 12 markers listed in table 3.

Table 4 shows markers that were found to be significantly deregulated in patients with NMO vs. a first cohort of patients with confirmed MS in the CIS or RRMS form.

TABLE 4
dmat_cis + rrms(2)_nmo

| | median g1 | median g2 | qmedian | ttest_rawp | AUC |
|---|---|---|---|---|---|
| hsa-miR-223-5p | 18.364372 | 1.149798 | 15.971831 | 0.000017 | 0.764463 |
| hsa-miR-6737-3p | 8.011134 | 1.149798 | 6.967430 | 0.000265 | 0.688017 |
| hsa-miR-3912-3p | 1.004049 | 1.004049 | 1.000000 | 0.000354 | 0.688017 |
| hsa-miR-5094 | 5.667004 | 1.004049 | 5.644153 | 0.000483 | 0.700413 |
| hsa-miR-6131 | 1.004049 | 1.004049 | 1.000000 | 0.000510 | 0.597107 |
| hsa-miR-6735-3p | 5.011134 | 69.421053 | 0.072185 | 0.000686 | 0.141529 |
| hsa-miR-6818-3p | 15.669028 | 1.004049 | 15.605847 | 0.000687 | 0.739669 |
| hsa-miR-1468-5p | 53.027328 | 109.070850 | 0.486173 | 0.000717 | 0.175620 |
| hsa-miR-379-3p | 1.004049 | 1.004049 | 1.000000 | 0.001128 | 0.727273 |
| hsa-miR-411-3p | 1.004049 | 1.004049 | 1.000000 | 0.002165 | 0.710744 |
| hsa-miR-301b | 1.004049 | 1.004049 | 1.000000 | 0.003110 | 0.628099 |
| hsa-miR-6505-3p | 6.009109 | 2.769231 | 2.169956 | 0.003202 | 0.607438 |
| hsa-miR-671-3p | 74.406883 | 128.995951 | 0.576816 | 0.003664 | 0.177686 |
| hsa-miR-3934-5p | 1.004049 | 1.004049 | 1.000000 | 0.003737 | 0.640496 |
| hsa-miR-1304-5p | 1.004049 | 1.004049 | 1.000000 | 0.004093 | 0.642562 |
| hsa-miR-4753-3p | 1.004049 | 1.004049 | 1.000000 | 0.004132 | 0.600207 |
| hsa-miR-127-3p | 95.415992 | 53.202429 | 1.793452 | 0.004686 | 0.739669 |
| hsa-miR-4775 | 1.004049 | 1.004049 | 1.000000 | 0.004942 | 0.652893 |
| hsa-miR-4755-3p | 2.684211 | 56.674089 | 0.047362 | 0.005600 | 0.162190 |
| hsa-miR-493-3p | 1.004049 | 1.004049 | 1.000000 | 0.005803 | 0.566116 |
| hsa-miR-451b | 1.004049 | 1.004049 | 1.000000 | 0.005868 | 0.634298 |
| hsa-miR-411-5p | 1.004049 | 1.004049 | 1.000000 | 0.005882 | 0.608471 |
| hsa-miR-548ac | 4.239879 | 1.004049 | 4.222782 | 0.005937 | 0.634298 |
| hsa-miR-4662a-5p | 1.004049 | 1.004049 | 1.000000 | 0.006201 | 0.586777 |
| hsa-miR-548q | 1.587045 | 2.635628 | 0.602151 | 0.006822 | 0.543388 |
| hsa-miR-409-5p | 1.004049 | 1.004049 | 1.000000 | 0.006923 | 0.625000 |
| hsa-miR-1908-3p | 1.004049 | 73.425101 | 0.013674 | 0.009125 | 0.242769 |
| hsa-miR-937-3p | 121.627530 | 50.028340 | 2.431173 | 0.010618 | 0.721074 |
| hsa-miR-651-5p | 1.004049 | 1.004049 | 1.000000 | 0.011427 | 0.636364 |
| hsa-miR-188-5p | 1.004049 | 1.149798 | 0.873239 | 0.011709 | 0.550620 |

Preferred combinations of markers to be used in the methods, kits or computer program products of the invention comprise or consist of the first 2, 3, 4, 5, 6, 7, 8, 9, 10, 11, or 12 markers listed in table 4.

In summary, a comprehensive analysis of miRNA expression in blood of NMO patients vs. controls and MS patients is shown, including CIS patients and RRMS patients. Applying NGS and microarray analyses a set of 88 miRNAs was identified, which were significantly deregulated. Subsets of miRNA markers were identified that allow differentiation between NMO and healthy controls or NMO and MS.

1. A method for diagnosing neuromyelitis optica, predicting risk of developing neuromyelitis optica, or predicting an outcome of neuromyelitis optica in a patient suffering from or at risk of developing neuromyelitis optica, said method comprising the steps of:
a) determining in a sample from said patient, the expression level of at least one miRNA selected from the group consisting of the miRNA species hsa-miR-6131, hsa-miR-127-3p, hsa-miR-181a-2-3p, hsa-miR-6775-3p, hsa-miR-454-5p, hsa-miR-6735-3p, hsa-miR-23b-3p, hsa-miR-6840-5p, hsa-miR-4301, hsa-miR-6798-3p, hsa-miR-6513-3p, hsa-miR-28-5p, hsa-miR-181b-5p, hsa-miR-943, hsa-miR-6501-5p, hsa-miR-128′7-5p, hsa-miR-3605-3p, hsa-miR-4448, hsa-miR-3127-3p, hsa-miR-942-5p, hsa-miR-6737-3p, hsa-miR-4755-3p, hsa-miR-3150a-5p, hsa-miR-6762-3p, hsa-miR-505-5p, hsa-let-7f-5p, hsa-miR-6819-3p, hsa-miR-127-3p, hsa-miR-223-5p, hsa-miR-3912-3p, hsa-miR-5094, hsa-miR-6818-3p, hsa-miR-1468-5p, hsa-miR-379-3p, hsa-miR-411-3p, hsa-miR-301b, hsa-miR-6505-3p, hsa-miR-671-3p, hsa-miR-3934-5p, hsa-miR-1304-5p, hsa-miR-4753-3p, hsa-miR-4775, hsa-miR-493-3p, hsa-miR-451b, hsa-miR-548ac, hsa-miR-4662a-5p, hsa-miR-548q, hsa-miR-409-5p, hsa-miR-1908-3p, hsa-miR-937-3p, hsa-miR-651-5p, hsa-miR-188-5p, hsa-miR-548b-5p, hsa-miR-4714-5p, hsa-miR-6735-3p, hsa-miR-4504, hsa-miR-4635, hsa-miR-548n, hsa-miR-3128, hsa-miR-421, hsa-miR-6783-5p, hsa-miR-3677-3p, hsa-miR-6737-3p, hsa-miR-486-3p, hsa-miR-7-5p, hsa-miR-548t-3p, hsa-miR-450b-5p, also listed in table 1;
b) comparing the pattern of expression level(s) determined in step a) with one or several reference pattern(s) of expression levels; and
c) diagnosing neuromyelitis optica, predicting risk of developing neuromyelitis optica, or predicting an outcome of neuromyelitis optica from the outcome of the comparison in step b).

2. A method of classifying a sample of a patient suffering from or at risk of developing neuromyelitis optica, said method comprising the steps of:
a) determining in a sample from said patient, the expression level of at least one miRNA selected from the group consisting of the miRNA species hsa-miR-6131, hsa-miR-127-3p, hsa-miR-181a-2-3p, hsa-miR-6775-3p, hsa-miR-454-5p, hsa-miR-6735-3p, hsa-miR-23b-3p, hsa-miR-6840-5p, hsa-miR-4301, hsa-miR-6798-3p, hsa-miR-6513-3p, hsa-miR-28-5p, hsa-miR-181b-5p, hsa-miR-943, hsa-miR-6501-5p, hsa-miR-1287-5p, hsa-miR-3605-3p, hsa-miR-4448, hsa-miR-3127-3p, hsa-miR-942-5p, hsa-miR-6737-3p, hsa-miR-4755-3p, hsa-miR-3150a-5p, hsa-miR-6762-3p, hsa-miR-505-5p, hsa-let-7f-5p, hsa-miR-6819-3p, hsa-miR-127-3p, hsa-miR-223-5p, hsa-miR-3912-3p, hsa-miR-5094, hsa-miR-6818-3p, hsa-miR-1468-5p, hsa-miR-379-3p, hsa-miR-411-3p, hsa-miR-301b, hsa-miR-6505-3p, hsa-miR-671-3p, hsa-miR-3934-5p, hsa-miR-1304-5p, hsa-miR-4753-3p, hsa-miR-4775, hsa-miR-493-3p, hsa-miR-451b, hsa-miR-548ac, hsa-miR-4662a-5p, hsa-miR-548q, hsa-miR-409-5p, hsa-miR-1908-3p, hsa-miR-937-3p, hsa-miR-651-5p, hsa-miR-188-5p, hsa-miR-548b-5p, hsa-miR-4714-5p, hsa-miR-6735-3p, hsa-miR-4504, hsa-miR-4635, hsa-miR-548n, hsa-miR-3128, hsa-miR-421, hsa-miR-6783-5p, hsa-miR-3677-3p, hsa-miR-6737-3p, hsa-miR-486-3p, hsa-miR-7-5p, hsa-miR-548t-3p, hsa-miR-450b-5p, also listed in table 1;
b) comparing the pattern of expression level(s) determined in step a) with one or several reference pattern(s) of expression levels; and;
c) classifying the sample of said patient from the outcome of the comparison in step b) into one of at least two classes indicative of a diagnosis of neuromyelitis optica, of predicting a risk of developing neuromyelitis optica, or of predicting an outcome of neuromyelitis optica.

3. The method according to claim 1, wherein the sample is selected from the group consisting of blood sample, serum sample, and plasma sample.

4. The method according to claim 1, comprising in step a) determining the expression level of at least one miRNA selected from the group consisting of the miRNA species hsa-miR-6131, hsa-miR-5094, hsa-miR-223-5p, hsa-miR-4753-3p, hsa-miR-6775-3p, hsa-miR-548b-5p, hsa-miR-3912-3p, hsa-miR-4714-5p, hsa-miR-6798-3p, hsa-miR-6501-5p, hsa-miR-454-5p, hsa-miR-6735-3p, hsa-miR-4504, hsa-miR-4301, hsa-miR-4635, hsa-miR-548n, hsa-miR-3128, hsa-miR-421, hsa-miR-1908-3p, hsa-miR-943, hsa-miR-6783-5p, hsa-miR-3677-3p, hsa-miR-3127-3p, hsa-miR-6737-3p, hsa-miR-486-3p, hsa-miR-7-5p, hsa-miR-548t-3p, hsa-miR-450b-5p, hsa-miR-486-3p, hsa-miR-505-5p as listed in table 2.

5. The method according to claim 1, comprising in step a) determining the expression level of the miRNA: hsa-miR-6131.

6. The method according to claim 1, further comprising determining from the outcome of step b) and/or c) whether a patient is suffering from or at risk of developing neuromyelitis optica versus multiple sclerosis.

7. The method according to claim 6, comprising in step a) determining the expression level of the at least one miRNA selected from the group consisting of the miRNA species hsa-miR-6131 and hsa-miR-127-3p.

8. The method according to claim 6, comprising in step a) determining the expression level of at least one miRNA selected from the group consisting of the miRNA species hsa-miR-6131, hsa-miR-127-3p, hsa-miR-181a-2-3p, hsa-miR-6775-3p, hsa-miR-454-5p, hsa-miR-6735-3p, hsa-miR-23b-3p, hsa-miR-6840-5p, hsa-miR-4301, hsa-miR-6798-3p, hsa-miR-6513-3p, hsa-miR-28-5p, hsa-miR-181b-5p, hsa-miR-181b-5p, hsa-miR-943, hsa-miR-3127-5p, hsa-miR-6501-5p, hsa-miR-1287-5p, hsa-miR-3605-3p, hsa-miR-4448, hsa-miR-3127-3p, hsa-miR-942-5p, hsa-miR-6737-3p, hsa-miR-4755-3p, hsa-miR-3150a-5p, hsa-miR-6762-3p, hsa-miR-505-5p, hsa-let-7f-5p, hsa-miR-6819-3p, as listed in table 3.

9. The method according to claim 6, comprising in step a) determining the expression level of at least one miRNA selected from the group consisting of the miRNA species hsa-miR-6131, hsa-miR-127-3p, hsa-miR-223-5p, hsa-miR-6737-3p, hsa-miR-3912-3p, hsa-miR-5094, hsa-miR-6735-3p, hsa-miR-6818-3p, hsa-miR-1468-5p, hsa-miR-379-3p, hsa-miR-411-3p, hsa-miR-301b, hsa-miR-6505-3p, hsa-miR-671-3p, hsa-miR-3934-5p, hsa-miR-1304-5p, hsa-miR-4753-3p, hsa-miR-4775, hsa-miR-4755-3p, hsa-miR-493-3p, hsa-miR-451b, hsa-miR-411-5p, hsa-miR-548ac, hsa-miR-4662a-5p, hsa-miR-548q, hsa-miR-409-5p, hsa-miR-1908-3p, hsa-miR-937-3p, hsa-miR-651-5p, hsa-miR-188-5p, as listed in table 4.

10. The method according to claim 6, comprising in step a) determining the expression level of at least one miRNA selected from the group consisting of the miRNA species hsa-miR-6131 and hsa-miR-127-3p, and at least one further miRNA selected from the group consisting of the miRNA species hsa-miR-181a-2-3p, hsa-miR-6775-3p, hsa-miR-454-5p, hsa-miR-6735-3p, hsa-miR-23b-3p, hsa-miR-6840-5p, hsa-miR-4301, hsa-miR-6798-3p, hsa-miR-6513-3p, hsa-miR-28-5p, hsa-miR-181b-5p, hsa-miR-943, hsa-miR-6501-5p, hsa-miR-1287-5p, hsa-miR-3605-3p, hsa-miR-4448, hsa-miR-3127-3p, hsa-miR-942-5p, hsa-miR-6737-3p, hsa-miR-4755-3p, hsa-miR-3150a-5p, hsa-miR-6762-3p, hsa-miR-505-5p, hsa-let-7f-5p, hsa-miR-6819-3p, hsa-miR-127-3p, hsa-miR-223-5p, hsa-miR-3912-3p, hsa-miR-5094, hsa-miR-6818-3p, hsa-miR-1468-5p, hsa-miR-379-3p, hsa-miR-411-3p, hsa-miR-301b, hsa-miR-6505-3p, hsa-miR-671-3p, hsa-miR-3934-5p, hsa-miR-1304-5p, hsa-miR-4753-3p, hsa-miR-4775, hsa-miR-493-3p, hsa-miR-451b, hsa-miR-548ac, hsa-miR-4662a-5p, hsa-miR-548q, hsa-miR-409-5p, hsa-miR-1908-3p, hsa-miR-937-3p, hsa-miR-651-5p, hsa-miR-188-5p, hsa-miR-548b-5p, hsa-miR-4714-5p, hsa-miR-6735-3p, hsa-miR-4504, hsa-miR-4635, hsa-miR-548n, hsa-miR-3128, hsa-miR-421, hsa-miR-6783-5p, hsa-miR-3677-3p, hsa-miR-6737-3p, hsa-miR-486-3p, hsa-miR-7-5p, hsa-miR-548t-3p, hsa-miR-450b-5p, as also listed in tables 1, 2, 3, or 4.

11. The method according to claim 1, wherein the determination of the expression level in step (a) is obtained by use of a method selected from the group consisting of a Sequencing-based method, an array-based method and a PCR-based method.

12. A kit for diagnosing neuromyelitis optica, predicting risk of developing neuromyelitis optica, or predicting an outcome of neuromyelitis optica in a patient suffering from or at risk of developing neuromyelitis optica, said kit comprising
means for determining in said sample from said patient, an expression level of at least one miRNA selected from the group consisting of hsa-miR-6131, hsa-miR-127-3p, hsa-miR-181a-2-3p, hsa-miR-6775-3p, hsa-miR-454-5p, hsa-miR-6735-3p, hsa-miR-23b-3p, hsa-miR-6840-5p, hsa-miR-4301, hsa-miR-6798-3p, hsa-miR-6513-3p, hsa-miR-28-5p, hsa-miR-181b-5p, hsa-miR-943, hsa-miR-6501-5p, hsa-miR-1287-5p, hsa-miR-3605-3p, hsa-miR-4448, hsa-miR-3127-3p, hsa-miR-942-5p, hsa-miR-6737-3p, hsa-miR-4755-3p, hsa-miR-3150a-5p, hsa-miR-6762-3p, hsa-miR-505-5p, hsa-let-7f-5p, hsa-miR-6819-3p, hsa-miR-127-3p, hsa-miR-223-5p, hsa-miR-3912-3p, hsa-miR-5094, hsa-miR-6818-3p, hsa-miR-1468-5p, hsa-miR-379-3p, hsa-miR-411-3p, hsa-miR-301b, hsa-miR-6505-3p, hsa-miR-671-3p, hsa-miR-3934-5p, hsa-miR-1304-5p, hsa-miR-4753-3p, hsa-miR-4775, hsa-miR-493-3p, hsa-miR-451b, hsa-miR-548ac, hsa-miR-4662a-5p, hsa-miR-548q, hsa-miR-409-5p, hsa-miR-1908-3p, hsa-miR-937-3p, hsa-miR-651-5p, hsa-miR-188-5p, hsa-miR-548b-5p, hsa-miR-4714-5p, hsa-miR-6735-3p, hsa-miR-4504, hsa-miR-4635, hsa-miR-548n, hsa-miR-3128, hsa-miR-421, hsa-miR-6783-5p, hsa-miR-3677-3p, hsa-miR-6737-3p, hsa-miR-486-3p, hsa-miR-7-5p, hsa-miR-548t-3p, hsa-miR-450b-5p, also listed in table 1, and
at least one reference pattern of expression levels for comparing with the expression level of the at least one miRNA from said sample.

13. A computer program product for diagnosing neuromyelitis optica, predicting risk of developing neuromyelitis optica, or predicting an outcome of neuromyelitis optica in a patient suffering from or at risk of developing neuromyelitis optica, comprising
means for receiving data representing an expression level of at least one miRNA in a patient sample selected from the group consisting of hsa-miR-6131, hsa-miR-127-3p, hsa-miR-181a-2-3p, hsa-miR-6775-3p, hsa-miR-454-5p, hsa-miR-6735-3p, hsa-miR-23b-3p, hsa-miR-6840-5p, hsa-miR-4301, hsa-miR-6798-3p, hsa-miR-6513-3p, hsa-miR-28-5p, hsa-miR-181b-5p, hsa-miR-943, hsa-miR-6501-5p, hsa-miR-1287-5p, hsa-miR-3605-3p, hsa-miR-4448, hsa-miR-3127-3p, hsa-miR-942-5p, hsa-miR-6737-3p, hsa-miR-4755-3p, hsa-miR-3150a-5p, hsa-miR-6762-3p, hsa-miR-505-5p, hsa-let-7f-5p, hsa-miR-6819-3p, hsa-miR-127-3p, hsa-miR-223-5p, hsa-miR-3912-3p, hsa-miR-5094, hsa-miR-6818-3p, hsa-miR-1468-5p, hsa-miR-379-3p, hsa-miR-411-3p, hsa-miR-301b, hsa-miR-6505-3p, hsa-miR-671-3p, hsa-miR-3934-5p, hsa-miR-1304-5p, hsa-miR-4753-3p, hsa-miR-4775, hsa-miR-493-3p, hsa-miR-451b, hsa-miR-548ac, hsa-miR-4662a-5p, hsa-miR-548q, hsa-miR-409-5p, hsa-miR-1908-3p, hsa-miR-937-3p, hsa-miR-651-5p, hsa-miR-188-5p, hsa-miR-548b-5p, hsa-miR-4714-5p, hsa-miR-6735-3p, hsa-miR-4504, hsa-miR-4635, hsa-miR-548n, hsa-miR-3128, hsa-miR-421, hsa-miR-6783-5p, hsa-miR-3677-3p, hsa-miR-6737-3p, hsa-miR-486-3p, hsa-miR-7-5p, hsa-miR-548t-3p, hsa-miR-450b-5p, also listed in table 1,
means for receiving data representing at least one reference pattern of expression levels for comparing with the expression level of the at least one miRNA from said sample,
means for comparing said data representing the expression level of the at least one miRNA in a patient sample, and
means for determining a diagnosis of neuromyelitis optica, a prediction of a risk of developing neuromyelitis optica, or a prediction of an outcome of neuromyelitis optica from the outcome of the comparison in step b).
